(19) United States
(12) Patent Application Publication
(10) Pub. No.: US 2024/0120081 A1
(43) Pub. Date: Apr. 11, 2024

(54) METHOD FOR TRACKING THE STATUS OF AT LEAST ONE MEDICAL DEVICE IN A DISTRIBUTED MEDICAL DEVICE SYSTEM, METHOD FOR RETRIEVING THE STATUS OF AT LEAST ONE MEDICAL DEVICE IN A DISTRIBUTED MEDICAL DEVICE SYSTEM, AND MEDICAL DEVICE

(71) Applicant: OLYMPUS WINTER & IBE GMBH, Hamburg (DE)

(72) Inventor: Jens KRÜGER, Hamburg (DE)

(73) Assignee: OLYMPUS WINTER & IBE GMBH, Hamburg (DE)

(21) Appl. No.: 18/243,373

(22) Filed: Sep. 7, 2023

Related U.S. Application Data
(60) Provisional application No. 63/413,328, filed on Oct. 5, 2022.

(51) Int. Cl. G16H 40/40

(52) U.S. Cl. CPC G16H 40/40

(57) ABSTRACT

A method for tracking the status of at least one medical device in a distributed medical device system includes a) determining an operational status of the at least one medical device, b) computing a digital representation of the operational status of the at least one medical device, and c) storing the digital representation in an electronic storage medium of the at least one medical device; wherein the method further d) comparing the digital representation of the operational status with a previously stored digital representation of a previously determined operational status of the at least one medical device, e) preparing a change report identifying any differences between the digital representation of the current status and the previously stored digital representation of the previously determined operational status, f) transmitting the change report to a status repository server, and g) storing of the change report in a database.

### FIELD

The present disclosure relates to medical devices and distributed medical device systems. More specifically, the present disclosure relates to methods of tracking or retrieving a status of at least one medical device in a distributed medical device system.

### BACKGROUND

In recent times, medical devices have greatly contributed to the capabilities of modern healthcare. Modern computer-controlled medical devices have been developed to provide a host of different diagnostic and therapeutic functions. However, the growing complexity of such computer-controlled medical devices makes servicing of the medical devices more difficult.

Computer-controlled medical devices usually comprise a medical function unit configured to execute one or more diagnostic or therapeutic functions in relation to a patient, a control unit configured to control the operation of the medical function unit, and a memory unit.

Distributed medical device systems have been developed to simplify servicing. In distributed medical device systems, one or more medical devices may be connected to a centralized server through a network. The medical devices of a distributed medical device system may transmit status messages to the centralized server, which may later be analysed by trained specialists to identify any issues in the operation of the medical devices.

In a known approach, the control unit of a medical device in a distributed medical device system is configured, or programmed, to transmit predetermined status information to the central server. In case of a defect, a malfunction, or any other issue with the medical device, service specialists may access the status information through the centralized server, and analyse the status information to identify the cause of the issue.

With the growing complexity of modern computer-controlled medical devices, the total volume of available status information increases, so that the volume of status messages also increases. At the same time, potential causes for any issues become difficult to predict, so that it is not always guaranteed that the status information available on the centralized server is sufficient for finding the true cause of an issue.

It would be beneficial to provide a method for tracking the status of a medical device, wherein all status information of the medical device would be available for analysis irrespective of any predetermined selection of status information to be transmitted to the server.

It would further be beneficial to provide a method for retrieving the status of a medical device, wherein the full status of the medical device could be obtained for analysis irrespective of any predetermined selection of status information to be transmitted to the server.

It would also be beneficial to limit total volume of status messages to be transferred in a distributed medical device system.

### SUMMARY OF THE DISCLOSURE

The present disclosure provides a method for tracking the status of at least one medical device in a distributed medical device system, wherein the distributed medical device system comprises: at least one medical device comprising a medical function unit, a control unit, and a memory unit; a status repository server, the status repository server being connected with the at least one medical device through a network; and a database; the method comprising the steps of a) determining, by the control unit of the at least one medical device, an operational status of the at least one medical device; b) computing, by the control unit of the at least one medical device, a digital representation of the operational status of the at least one medical device; and c) storing, by the control unit of the respective medical device, the digital representation in the memory unit of the at least one medical device; wherein the method further comprises the steps of d) comparing, by the control unit of the respective medical device, the digital representation of the operational status with a previously stored digital representation of a previously determined operational status of the at least one medical device; e) preparing, by the control unit of the at least one medical device, a change report identifying any differences between the digital representation of the current status and the previously stored digital representation of the previously determined operational status; f) transmitting, by the control unit of the at least one medical device, the change report to the status repository server; and g) storing, by the status repository server, of the change report in the database.

With the method according to the present disclosure, it is no longer necessary to define, during design of a medical device, which kind of status data is to be reported in a distributed medical device system. Instead, the complete status of the medical device can always be reproduced from the change reports. At the same time, the total volume of status data messages to be transmitted can be kept relatively low, as only incremental changes of the status need to be reported.

The steps a) to g) may be repeated in a loop while the at least one medical device is operating.

The step of computing the digital representation of the operational status of the at least one medical device may include writing at least one of system events, status messages, and error messages to at least one log file. The at least one medical device may comprise a user interface, and the step of computing the digital representation of the operational status of the at least one medical device may include writing user input received through the user interface to at least one log file. The step of storing the digital representation in the memory unit may include writing the at least one log file to a file system of the memory unit. The step of computing the digital representation of the operational status of the at least one medical device may include writing at least one operational parameter of the medical device to at least one configuration file. The step of storing the digital representation in the memory unit may include writing the at least one configuration file to a file system of the memory unit.

The step of comparing the digital representation of the operational status with a previously stored digital representation of a previously determined operational status may include scanning the file system of the memory unit for at least one of newly added files, deleted files, and changed files. The step of preparing the change report may comprise at least one of: writing an identifier of any newly added file, deleted file, and changed file to the change report; writing the content of any newly added file to the change report; and writing the content difference of any changed file to the change report. The method may further comprise a step of writing at least one of a time stamp, a medical device identifier, and a unique change identifier to the change report.

The method may further comprise a step of compressing the change report before the step of transmitting the change report. The method may further comprise a step of encrypting the change report before the step of transmitting the change report.

The method may use revision control systems like GIT or SVN, or similar approaches.

The present disclosure further provides a method for retrieving the status of at least one medical device in a distributed medical device system, wherein the distributed medical device system comprises: at least one medical device; a status repository server, the status repository server being connected with the at least one medical device through a network; and a database; the method comprising the steps of retrieving, through the status repository server, a digital representation of an initial status of the at least one medical device from the database; retrieving, through the status repository server, a sequence of change reports for the at least one medical device from the database; and sequentially applying the changes listed in the retrieved sequence of change reports to the digital representation of the initial status, to obtain a digital representation of the current status of the at least one medical device.

The disclosure further provides a medical device, comprising a medical function unit, a control unit, and a memory unit, wherein the control unit of the medical device is configured to execute steps a) to f) of the method provided by the disclosure.

### BRIEF DESCRIPTION OF THE DRAWINGS

The subject of the present disclosure is further described with relation to a number of exemplary drawings. The drawings and any embodiments shown therein are provided only for better understanding of the disclosure without limiting the scope of the invention, which is defined in the appending claims.

The drawings show:

FIG. 1: a distributed medical device system;

FIG. 2: a medical device of a distributed medical device system;

FIG. 3: a method for tracking the status of a medical device;

FIG. 4: a method for retrieving the status of a medical device.

### DETAILED DESCRIPTION

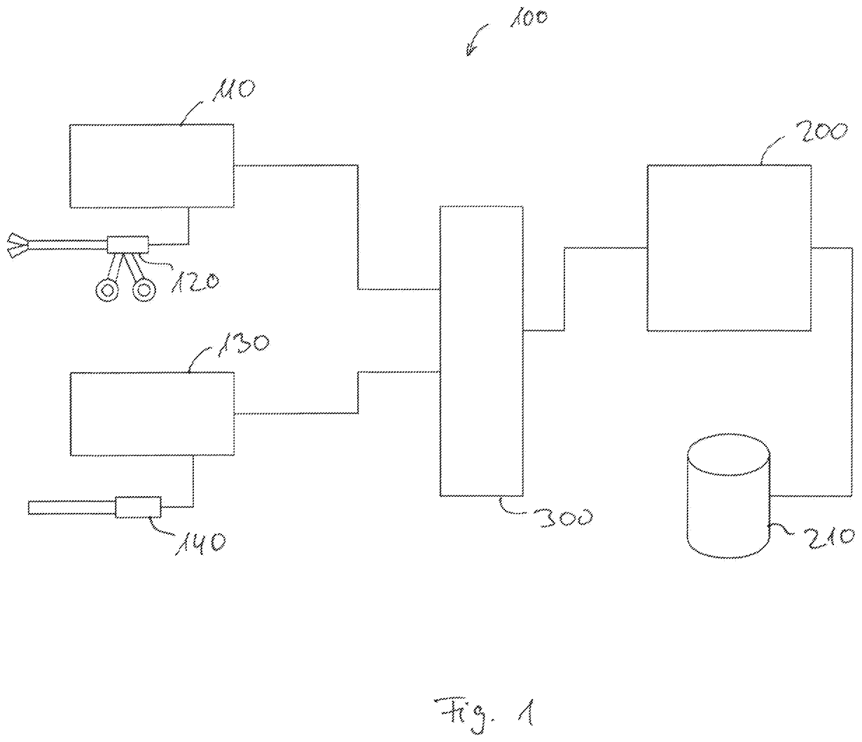

FIG. 1 shows an exemplary distributed medical device system 100. The system 100 comprises a first medical device 110. In the present example, the first medical device 110 is an electrosurgical generator, connected to electrosurgical forceps 120. The system 100 further comprises a second medical device 130. In the present example, the second medical device is an endoscope camera controller, connected to a surgical endoscope 140.

The system 100 also comprises a status repository server 200. The status repository server is connected to a database 210. The medical devices 110, 130 and the status repository server 200 are connected through a network 300, like a hospital intranet or the internet.

The status repository server 200 may be a standard computer, running a standard operating system like Windows™, LINUX™, or MacOS™. The Database 210 may be hosted on the status repository server 200 and is depicted as a separate entity only for better understanding.

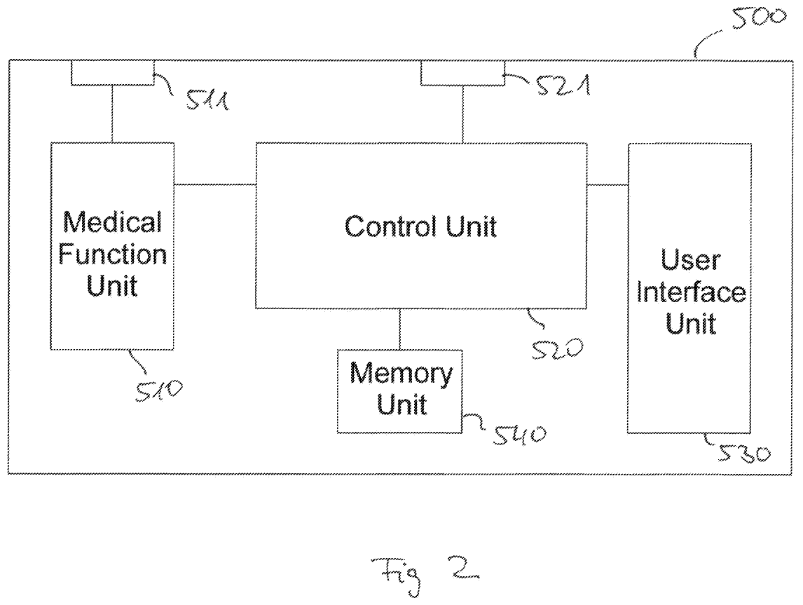

FIG. 2 shows a medical device 500 in more detail. The medical device 500 may be one of the medical devices 110, 130 shown in FIG. 1. The medical device 110 comprises a medical function unit 510, a control unit 520, a user interface unit 530, and a memory unit 540. The medical function unit 510 is designed to provide a particular medical functionality characteristic of the medical device 500. In case of an electrosurgical generator, the medical function unit 510 comprises circuitry for generating, controlling, and outputting electrosurgical signals. Such circuitry may comprise an RF oscillator, current and/or voltage sensors, feedback control circuitry, isolation transformers, and the like.

In case of an endoscope camera controller, the medical function unit 510 may comprise video control circuitry for performing shutter control, illumination control, focus control, or the like. The medical function unit may further comprise video processing circuitry.

In case of a medical pump like an irrigation pump, suction pump, insufflator pump, or the like, the medical function unit 510 may comprise a mechanical pump like a peristaltic pump, a piston pump, or the like, and pressure and/or volume flow sensors, turbidity sensors, or the like.

The medical function unit 510 is connected to an interface 511. The interface 511 can be used to connect a medical instrument (not shown) to the medical device 500. Depending on the type of the medical device 500, the interface 511 may include a monopolar or bipolar socket for connecting an electrosurgical instrument like the forceps 120, a camera socket for connecting a video device like the endoscope 140, and/or fluid connectors for connecting suction and/or irrigation instruments.

The control unit 520 is provided for controlling the function of the medical function unit 510. In this context, the term “controlling the function” is meant to include one or more of triggering the medical function unit 510 to initiate or terminate a specific procedure, providing operational parameters to the medical function unit 510 for defining the specific medical procedure, monitoring a status of the medical function unit 510, monitoring the progress of the specific medical procedure, and the like.

The control unit 520 may be implemented with a conventional computer architecture. The control unit 520 may comprise one or more processors, memory devices, bus devices, and the like. The control unit 520 may be operating with a standard operating system like Windows™, LINUX™, MacOS™, or the like, or with a specialized operating system for medical devices. The control unit 520 may communicate with the medical function unit 510 through a standard bus system like RS232, I²C, fieldbus, or the like.

The control unit 520 may be connected to a data interface 521. The data interface 521 may include one or more of a wired network interface, a wireless network interface, a Bluetooth™ interface, or the like. The control unit 520 may use the data interface to send and/or receive status data, service data, software updates, and the like. The data interface 521 may be used to connect the medical device 500 to the status repository server 200 through the network 300, as shown in FIG. 1.

The user interface unit 530 serves for outputting status information to a user, and for allowing a user to input parameters and commands. Therefore, the user interface 520 may include on or more output devices like a monitor, indicator lamps, loudspeakers or the like, and one or more input devices like a keyboard, switches, buttons, and a graphical input device like a mouse or a touchpad, and the like. The user interface unit 530 may include a combined input/output device like a touch screen. The user interface unit 530 may be an integral part of the medical device 500. The user interface unit 530 may be fully or partially separate from the medical device 500. The user interface unit 530 may include a detachable remote operating panel. The control unit 520 may communicate with the user interface unit 530 through a standard bus system like RS232, I²C, fieldbus, or the like.

The memory unit 540 may include any known type of data storing unit including, but not limited to, a hard disk drive (HDD), a solid state disk (SSD), a flash disk drive, and the like. The control unit 520 may communicate with the memory unit 540 through a standard interface like IDE, P-ATA, S-ATA, SCSI, SAS, or the like. The control unit 520 may communicate with the memory unit for temporarily or permanently storing status information of the control unit 520, the medical function unit 510, and/or the user interface unit 530. The Memory unit may store program instructions to be read out and executed by the one or more processors of the control unit 520. Information stored in the memory unit 540 may be organized in a file system like ISO 9660, UDF, FAT, exFAT, NTSF, ext2, or the like.

During operation of the medical device 500, the control unit 520 is permanently collecting status information from the medical function unit 510 and the user interface unit 530, including sensor logs, error logs, user settings, user input, and the like, and stores such status information on the memory unit 540. The body of status data stored on the memory unit 540 can be used for comprehensively describing the current status of the medical device 500, and can therefore be considered a digital representation of the status of the medical device 500.

For easily assessing the current status of the medical device, some medical devices are configured to collect a predefined selection of status information data in a separate log file, which can be accessed through the data interface 521. The status information data stored in the log file can then be analysed in case of a malfunction, in order to identify the cause of the error. However, with modern medical devices getting more and more complex, it is difficult to predict what kind of status information data will be required for this purpose.

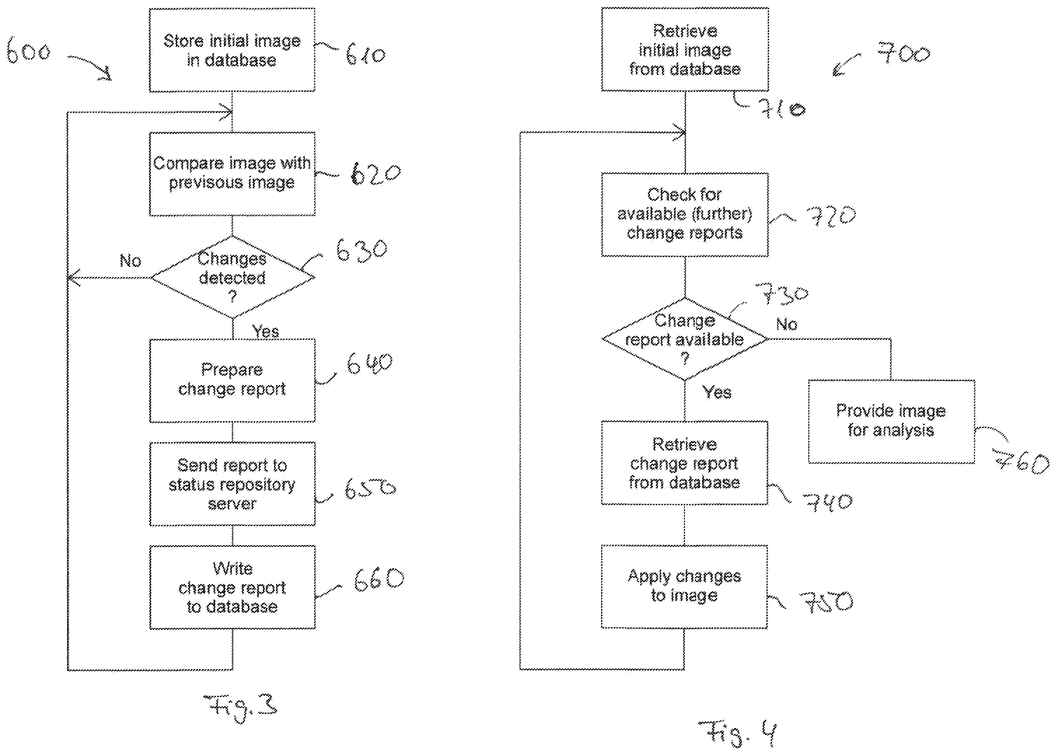

In the medical device according to the present disclosure, the control unit 520 is not configured to store predefined status information data in a separate log file. Instead, the control unit 520 is configured to always provide, in combination with the status repository server 200 and the database 210 described with respect to FIG. 1, a comprehensive description of the status of the medical device 500. A method 600 therefore is shown in FIG. 3.

For this purpose, in Step 610 the contents of the memory unit 540 upon factory delivery of medical device 500 are deposited in the database 210 as a representation of the initial status of the medical device 500. This digital representation can include an image of the file system of the memory unit 540. At the time of factory deliver, the file system may hold a number of configuration files, in which initial operating parameters of the medical function unit are stored. Other configuration files may contain a list of registered users provided by the user interface device, which may only contain a default user at factory delivery, a list of connected medical instruments and related settings, which may be empty at factory delivery, and the like. The file system may further hold a number of log files for sensor data like device temperature data, current and/or voltage output of an electrosurgical generator, flow rate and/or accumulated volume of a surgical pump, or the like. Such log filed may be empty at factory delivery, or may contain data logged during testing of the medical device 500 before delivery. The file system may further comprise a log file for system event messages like warning messages, error messages, or the like, and a log file for user instructions input through the user interface unit 530.

After factory delivery, the control unit 520 is programmed to repeatedly compare the current contents of the memory unit 540 with the contents of the memory unit at a previous run in step 620. Methods for identifying changes in a file system are known to the skilled person and need not be described in detail here.

In branching step 630, if any changes are detected, the control unit 520 is further programmed to proceed to step 640, to prepare a change report identifying the detected changes. If no changes are detected, the control unit 520 is programmed to return to step 620.

Preparing the change report may include compiling a list of any files in the file system which have been changed, newly created, or deleted in the file system. As an example, when a new user has been registered in the user interface unit 530, a configuration file comprising a list of registered users may have changed, and a new file with preferred settings of the new user may have been created. Similarly, if an electrosurgical instrument has been used with the medical device 500 in a procedure, a status file containing usage data of the electrosurgical instrument may have changed. On the other hand, if an electrosurgical instrument has been connected to the medical device 500 for the first time, a new status file may have been created in the file system stored on the memory unit 540, for storing usage information of the electrosurgical instrument. Examples of deleted files are configuration files with preferred setting of users being deleted from the list of registered users (e.g. because the user has transferred to another department, left the facility, or the like), and usage status files of electrosurgical instruments that have reached or exceeded their maximum allowed usage, and the like. Changes in other log or configuration files may occur if a user switches the user interface unit 530 from one display screen to another one, changes parameter settings, or the like. If a system error occurs in the medical function unit 510 or the control unit, an error log file may be updated and therefore change.

Along with the list of files that have been changed, added, or deleted, the change report preferably contains the added or changed content of the affected files. The change report may further comprise a time stamp showing the time of creation of the change report, a medical device identifier unambiguously identifying the medical device 500, and a change identifier. The change identifier may be a hash value of the change report or a unique token.

After compiling the change report, the control unit 520 sends the change report to the status repository server 200 through the data interface 521 and the network 300 in step 650. In the following step 660, the status repository server 200 stores the change report in the database 210.

As with the described method, only incremental changes of the digital representation of the status of medical device 500 need to be communicated, the data traffic volume can be kept low, while at the same time a full representation of the status is always available for analysis at the status repository server.

For further reducing the traffic volume, the change reports may be compressed before sending. In some cases, the change reports may comprise sensitive data, like patient data or personal data from user list entries. For protecting such sensitive data from illicit access, the change reports may further be encrypted before transmission. Encryption may be achieved with any suitable encryption algorithm known to the skilled person. The status repository server may decompress and/or decrypt the change reports as needed before storing them in the database.

The network connection of the medical device 500 may also be used for distributing parameter and software updates to the medical device 500. This can be either a “push” service, where a server actively notifies the medical device 500 of available updates, or a “pull” service, where the medical device 500 repeatedly queries a server whether or not updates are available. In either case, the medical device 500 can then download available updates from the server. The server may be the status repository server or a separate, dedicated update server.

In some instances, it may be desirable to retrieve the status of the medical device 500. For example, if a user of the medical device 500 encounters a problem like a malfunction, they may contact the service department of the producer of the medical device 500 and report the issue. A service expert may then want to know the status of the medical device 500 before the issue arose.

Therefore, the service expert may use the status repository server 200 to retrieve a digital representation of the status of the medical device 500 from the database 210. A method 700 therefore is shown in FIG. 4.

In a first step 710, the status repository server 200 retrieves the initial contents of the memory unit 540 from the database 210. The initial contents may include an image of the file system of memory unit 540. The status repository server 200 may use the image to virtually reproduce the memory unit 540 at the time of factory delivery of the medical device 500.

In a second step 720, the status repository server 200 checks the database 210 for available change reports for the medical device 500.

If at least one change report is available, branching step 730 passes to step 740, where the change report is retrieved from the database 210. In a further step 750, the change report is then applied to the virtual reproduction, or image, of the memory unit 540. Here, the “application” of the change report is to be understood as reproducing the individual addition, deletion, or change of files in the file system of the memory unit 540, so that the image of the memory unit 540 after application of the change report corresponds to the contents of the memory unit 540 at the time the respective change report was created.

The process then loops back to step 720, to check whether further change reports are available in the database 210.

After all available change reports have been retrieved and applied, the branching step 730 passes to step 760, in which the final image is provided for analysis. At this time, the image corresponds to the contents of the memory unit 540 at the time the last change report has been sent, so that a service expert is able to fully comprehend the status of the medical device 500 at this time.

For further analysis, it is also possible to reproduce the status of the medical device 500 at different times, for example after each loop of the process 700. In this way, a time-lapse analysis of the status of the medical device 500 can be obtained. With such time-lapse analysis, dynamic effects resulting in issues, errors, or malfunctions of the medical device 500, can be identified.

In a distributed medical device system comprising a plurality of similar medical devices, machine learning algorithms may be applied to identify and/or predict any issues or errors in one or more of the medical devices.

In the methods 600, 700 described above, known algorithms from revision control systems like GIT, SVN, or the like, may be applied.

1. A method for tracking the status of at least one medical device in a distributed medical device system, wherein the distributed medical device system comprises:
at least one medical device comprising a medical function unit a control unit, and a memory unit;
a status repository server, the status repository server being connected with the at least one medical device through a network; and
a database;
the method comprising the steps of
a) determining, by the control unit of the at least one medical device, an operational status of the at least one medical device,
b) computing, by the control unit of the at least one medical device, a digital representation of the operational status of the at least one medical device, and
c) storing, by the control unit of the respective medical device, the digital representation in the memory unit of the at least one medical device;
wherein the method further comprises the steps of
d) comparing, by the control unit of the respective medical device, the digital representation of the operational status with a previously stored digital representation of a previously determined operational status of the at least one medical device,
e) preparing, by the control unit of the at least one medical device, a change report identifying any differences between the digital representation of the current status and the previously stored digital representation of the previously determined operational status,
f) transmitting, by the control unit of the at least one medical device, the change report to the status repository server, and
g) storing, by the status repository server, of the change report in the database.

2. The method of claim 1, wherein the steps a) to g) are repeated in a loop while the at least one medical device is operating.

3. The method of claim 1, wherein the step of computing the digital representation of the operational status of the at least one medical device includes writing at least one of system events, status messages, and error messages to at least one log file.

4. The method of claim 1, wherein the at least one medical device comprises a user interface, and wherein the step of computing the digital representation of the operational status of the at least one medical device includes writing user input received through the user interface to at least one log file.

5. The method of claim 3, wherein the step of storing the digital representation in the memory unit includes writing the at least one log file to a file system of the memory unit.

6. The method of claim 1, wherein the step of computing the digital representation of the operational status of the at least one medical device includes writing at least one operational parameter of the medical device to at least one configuration file.

7. The method of claim 6, wherein the step of storing the digital representation in the memory unit includes writing the at least one configuration file to a file system of the memory unit.

8. The method of claim 5, wherein the step of comparing the digital representation of the operational status with a previously stored digital representation of a previously determined operational status includes scanning the file system of the memory unit for at least one of newly added files, deleted files, and changed files.

9. The method of claim 8, wherein the step of preparing the change report comprises at least one of:
writing an identifier of any newly added file, deleted file, and changed file to the change report,
writing the content of any newly added file to the change report, and
writing the content difference of any changed file to the change report.

10. The method of claim 9, further comprising a step of writing at least one of a time stamp, a medical device identifier, and a unique change identifier to the change report.

11. The method of claim 9, further comprising a step of compressing the change report before the step of transmitting the change report.

12. The method of claim 9, further comprising a step of encrypting the change report before the step of transmitting the change report.

13. The method of claim 1, using revision control systems like GIT or SVN.

14. A method for retrieving the status of at least one medical device in a distributed medical device system, wherein the distributed medical device system comprises:
at least one medical device;
a status repository server, the status repository server being connected with the at least one medical device through a network; and
a database;
the method comprising the steps of
retrieving, through the status repository server, a digital representation of an initial status of the at least one medical device from the database;
retrieving, through the status repository server, a sequence of change reports for the at least one medical device from the database; and
sequentially applying the changes listed in the retrieved sequence of change reports to the digital representation of the initial status, to obtain a digital representation of the current status of the at least one medical device device.

15. A medical device, comprising a medical function unit a control unit, and a memory unit, wherein the control unit of the medical device is configured to execute steps a) to f) of the method according to claim 1.
